(19) United States
(12) Patent Application Publication
(10) Pub. No.: US 2012/0203254 A1
(43) Pub. Date: Aug. 9, 2012

(54) Allogenic Vessel Nerve Conduit

(76) Inventor: David Tak Wai Chiu, New York, NY (US)

(21) Appl. No.: 13/360,795

(22) Filed: Jan. 29, 2012

Related U.S. Application Data
(60) Provisional application No. 61/439,967, filed on Feb. 7, 2011.

(51) Int. Cl. A61B 17/00

(52) U.S. Cl. 606/152

(57) ABSTRACT

The present invention relates to a method for repairing a peripheral nerve injury. The method comprises providing an immunologically inactive allogenic vessel at a peripheral nerve injury site and routing nerve tissue through the vessel lumen to bridge the peripheral nerve injury site.

This invention claims priority to U.S. provisional patent application 61/439,967, filed on Feb. 7, 2011.

### FIELD OF THE INVENTION

The present invention relates to a method for repairing a peripheral nerve injury. The method comprises providing an immunologically inactive allogenic vessel at a peripheral nerve injury site and routing nerve tissue through the lumen of the vessel to bridge the peripheral nerve injury site.

### BACKGROUND

Peripheral nerve defects can occur as a result of trauma or tumor resection. A surgeon faced with such a problem must repair the defect in order to restore nerve function. Ideally, the nerve is repaired by direct coaptation through stump mobilization or rerouting. When these techniques fail to provide a tensionless reunion of the divided stumps other techniques must be used. Currently, the most commonly applied approach is the autogenous nerve graft method. However, donor sites for autogenous nerve graft are limited; there exists a need for other forms of nerve graft.

Applicability of using a segment of autogenous vein to bridge a nerve gap as an interposition conduit was ascertained experimentally in 1982 (see Chiu et al., Autogenous vein graft as a conduit for nerve regeneration. Surg. Forum 1980:31:550, Chiu et al., Autogenous vein graft as a conduit for nerve regeneration. Surgery 1982:91:226-233, Chiu et al., Comparative electrophysiological evaluation of nerve grafts and autogenous vein graft as nerve conduits: an experimental study. Journal of reconstructive microsurgery 1988; 4940:303-309), and clinically in 1990 (Chiu et al., Prospective clinical evaluation of autogenous vein grafts used as a nerve conduit for distal sensory nerve defects of 3 cm or less. Plast. Reconstruct Surg. 1990; 86:928-934). Over the past two decades, autogenous venous nerve conduit has evolved as a conventional method of reconstructing peripheral nerve with nerve gaps smaller than 3 cm. While an autogenous vein graft is useful to bridge a nerve gap, the technique requires harvesting the vein graft surgically, an added procedure that takes time and results in a scar at the donor site. Use of an allogenic vessel to bridge a nerve gap will obviate such disadvantages.

### SUMMARY OF THE INVENTION

In one aspect, the invention is a method for repairing a peripheral nerve at an injury site. The method comprises providing an immunologically inactive allogenic vessel at the peripheral nerve injury site and routing nerve tissue through the lumen of the vessel to bridge the peripheral nerve gap. The allogenic vessel may be rendered immunologically inactive by freezing and thawing, by formalin treatment, or by radiation treatment. Furthermore, the immunologically inactive allogenic vessel may be potentiated by adding to its lumen a nerve regeneration enhancing agent, such as neurotrophic growth factor.

### DETAILED DESCRIPTION OF THE INVENTION

The present method is a novel modification of the autogenous vein graft method. The method comprises providing an immunologically inactive allogenic vessel at a peripheral nerve injury site and routing nerve tissue (such as nerve autograft) through the lumen of the vessel to bridge the peripheral nerve injury site.

The advantages of the method include that there is no need to harvest the vessel surgically, therefore, eliminating any surgical morbidity and other potential complications. Additionally, such a construct allows for the bioengineering or restructuring of the internal milieu of the conduit, for example by the addition of a nerve regeneration enhancing substance, such as a nerve growth factor.

As a first step an allogenic vein or artery graft is rendered immunologically inactive. After rendering the allogenic vein or artery immunologically inactive, the allogenic vessel can be cold-stored for later use. When a peripheral nerve injury has to be repaired, the immunologically inactive allogenic vessel is thawed and used as a nerve conduit. The immunologically inactive conduit provides a protected passage of space for nerve regeneration and a permanent barrier against fibroblast invasion.

To prepare the immunologically inactive vessel conduit, a formalin treated vessel had been found to be better as a bridge graft than fresh vessels. A simple approach to de-immunize the allograft is storing the graft in Ringer's solution at 2° Centigrade. Another approach is to treat the graft with formalin or radiation. Nerve homograft has been shown to function well as an interposition graft after being stored in 0.9% saline at 37° Centigrade for as long as 3 weeks, or after being stored in Ringer's solution at 2° Centigrade for 21 days. See Sanders, F K., The repair of large gaps in the peripheral nerves, Brain, 65 (1942), page 281-337, herein incorporated by reference.

For a major peripheral nerve, the fascicles can be divided into subgroups and bridged separately.

The practice of the invention is illustrated by the following non-limiting examples.

### Example

In this Example, a surgeon is treating a patient with a 3 cm defect of the radial palmar digital nerve. The surgeon sends for a cold-preserved saphenous vein graft or ulnar artery graft which has been rendered immunologically inactive. It is thawed and used as a nerve conduit to restore the continuity of the injured nerve. Techniques of de-immunization include, but are not limited, to freezing and thawing cycles, formalization and radiation.

Although specific methods have been described, it will be appreciated that various modifications and changes may be made therein without departing from the spirit and scope of the invention as defined by the following claims.

I claim:

1. A method for repairing a peripheral nerve at a peripheral nerve injury site, the method comprising providing an immunologically inactive allogenic vessel at the peripheral nerve injury site and routing nerve tissue through the lumen of the vessel to bridge the peripheral nerve injury site.

2. The method of claim 1, wherein the allogenic vessel has been rendered immunologically inactive by a freeze and thaw cycle.

3. The method of claim 1, wherein the allogenic vessel has been rendered immunologically inactive by formalin treatment.

4. The method of claim 1, wherein the allogenic vessel has been rendered immunologically inactive by radiation treatment.

5. The method of claim 1, wherein the immunologically inactive allogenic vessel contains a neurotrophic growth factor.

6. The method of claim 1, wherein the nerve tissue placed in the vessel lumen is nerve autograft.

7. A method for decreasing scarring at a peripheral nerve injury site, the method comprising providing an immunologically inactive allogenic vessel at the peripheral nerve injury site and routing nerve tissue through the lumen of the vessel to bridge the peripheral nerve injury site.

8. The method of claim 7, wherein the allogenic vessel has been rendered immunologically inactive by a freeze and thaw cycle.

9. The method of claim 7, wherein the allogenic vessel has been rendered immunologically inactive by formalin treatment.

10. The method of claim 7, wherein the allogenic vessel has been rendered immunologically inactive by radiation treatment.

11. The method of claim 7, wherein the immunologically inactive allogenic vessel contains a neurotrophic growth factor.

12. The method of claim 7, wherein the nerve tissue placed in the vessel lumen is nerve autograft.
